(19) United States
(12) Patent Application Publication
(10) Pub. No.: US 2010/0217391 A1
(43) Pub. Date: Aug. 26, 2010

(54) TENSIONING BONE IMPLANT DEVICE

(76) Inventor: Amy L. Ladd, Stanford, CA (US)

Correspondence Address: KENYON & KENYON LLP, ONE BROADWAY, NEW YORK, NY, 10004, US

(21) Appl. No.: 12/611,700

(22) Filed: Nov. 3, 2009

Related U.S. Application Data
(60) Provisional application No. 61/208,279, filed on Feb. 21, 2009.

(51) Int. Cl. A61F 2/28

(52) U.S. Cl. 623/16.11

(57) ABSTRACT

Described is a tensioning bone implant device, a method of using the tensioning bone implant device, and a method of manufacturing the tensioning bone implant device. The tensioning bone implant device may include a resilient hinge, a first tensioning arm attached to one end of the hinge, and a second tensioning arm attached to an other end of the hinge, in which each of the hinge, the first tensioning arm, and the second tensioning arm includes at least one loop formed from wire.

### CROSS-REFERENCE TO RELATED APPLICATIONS

This application claims the benefit of U.S. Provisional Patent Application No. 61/208,279, filed on Feb. 21, 2009, which is expressly incorporated herein in its entirety by reference thereto.

### FIELD OF THE INVENTION

The present invention relates to a tensioning bone implant device, a method of using the bone implant device, and a method of manufacturing the bone implant device.

### BACKGROUND INFORMATION

Bone or fracture voids may occur in many different types of bones in many different ways. For example, an unstable distal radius fracture is common especially in the endemic osteoporotic populations of North America, Europe, Asia, and Australia. This type of low energy fracture may be sustained by a fall on an outstretched hand. The classic, osteopenic osteoporotic fragility fracture is extra-articular or includes a simple intra-articular component, i.e., the fracture is primarily outside of a joint or may include a simple component within the joint. The fracture may result in dorsal comminution, loss of radial height, loss of volar tilt, radial shift, and shortening. In this regard, dorsal comminution refers to pulverization of the bone in the wrist in the direction of the back of the hand, loss of radial height refers to loss of height in the wrist on the side near the thumb, loss of volar tilt refers to loss of tilt in the wrist in the direction of the palm of the hand, and radial shift refers to shift of the wrist towards the side of the thumb. In addition, poor bone mineral quality and the degree of comminution, especially with proximal extension on the radial column, may render this fracture unstable, such that closed treatment alone may be insufficient.

Further, the forces experienced by the wrist during daily activities are primarily compression, e.g., digital motion, and shear/torsion, e.g., forearm rotation. Fracture, e.g., catastrophic collapse, occurs typically in tension, thereby creating a relatively transverse fracture across the metaphysis, the metaphysis being the part of a bone between the shaft of the bone, i.e. diaphysis, and the end of the bone, i.e., epiphysis. The position of the wrist, the forces applied, and the bone quality may determine other components of the fracture, such as, for example, extension into the joint, extension into the diaphysis, and more oblique components from torsional forces.

Reduction, i.e., architectural restoration, of a simple but unstable fracture may be obtained through a variety of means. Although there has been a historical preference for nonoperative treatment, more invasive treatments intended to restore cortical, i.e. external or surface, integrity have historically included pins and plaster techniques, external fixation, and cross metaphyseal pinning. Later treatment techniques have included dorsal plating systems that address the radial column, and volar plate fixation. Examples of dorsal plating systems include, e.g., Forte Zimmer low profile plate or Synthes pi plate. The more rigid construct required for volar fixation, given its application on the compression side of the radius, has been purportedly outweighed by soft tissue coverage of the volar plate not afforded by dorsal plating systems.

Although plating systems may address cortical reconstitution, they do not address metaphyseal voids that are formed when osteopenic/osteoporotic bone collapses. Further, rigid volar plates may not adequately overcome the loss of cancellous bone in the metaphysis when significant comminution and severe loss of bony architecture has occurred. To fill these metaphyseal voids, patients' autograft bone, banked allograft bone, and/or synthetic fillers, e.g., calcium phosphate or calcium sulfate, may be used. Moreover, although PMMA (polymethylmethacrylate) cement has historically been used as a void filler, this material is rarely used in radius fractures since biologic and biologically active alternatives are preferred.

Plating systems and volar plate fixation may be more substantial and invasive than a patient's bone or fracture void and comorbidities may warrant. Accordingly, there is a need for a device that addresses both metaphyseal and cortical reconstitution, restores stability, and causes minimal soft tissue invasion and bony disruption during implantation.

### SUMMARY

Example embodiments of the present invention provide a tensioning bone implant device, a method of using the bone implant device, and a method of manufacturing the bone implant device.

According to example embodiments of the present invention, a tensioning bone implant device may include a resilient hinge, a first tensioning arm attached to one end of the hinge, and a second tensioning arm attached to an other end of the hinge, in which each of the hinge, the first tensioning arm, and the second tensioning arm includes at least one loop formed from wire.

The hinge may be configured to bias the first tensioning arm and the second tensioning arm away from each other. The hinge, the first tensioning arm, and the second tensioning arm may be formed from a single wire, or from multiple wires joined together. The hinge may include at least a single loop. The first tensioning arm may include at least a single loop, and the second tensioning arm may also include at least a single loop.

A plane defined by the at least one loop of the first tensioning arm may be substantially parallel to a plane defined by the at least one loop of the second tensioning arm. A plane defined by the at least one loop of the hinge may be substantially perpendicular to each of a plane defined by the at least one loop of the first tensioning arm and a plane defined by the at least one loop of the second tensioning arm.

The hinge, the first tensioning arm, and the second tensioning arm together may substantially form one of a trapezoidal, pyramidal, triangular, conical, oblong, and ovoid shape.

The wire may be composed of a biocompatible material. The biocompatible material may be at least one of stainless steel, nitinol, chrome-moly, other biocompatible metals, PEEK (polyaryletheretherketone), other biocompatible polymers and plastics, and other memory materials.

The device may further include a first locking element, e.g., a hook or other feature, on the first tensioning arm at an end opposite the hinge. The device may further include a second locking element, e.g., a hook or other feature, on the second tensioning arm at an end opposite the hinge. The first locking element and the second locking element may be configured to lock together to constrain a maximum distance between the first tensioning arm and the second tensioning arm.

The device may further include a first seating element on the first tensioning arm at an end opposite the hinge, the first seating element configured to seat the first tensioning arm in bone on one side of a bone or fracture void. The device may further include a second seating element on the second tensioning arm at an end opposite the hinge, the second seating element configured to seat the second tensioning arm in bone on an other side of a bone or fracture void.

The device may be configured to be implanted with minimal soft tissue invasion and bony disruption. The device may be configured to restore metaphyseal and cortical collapse of a bone or fracture void. The device may be configured to directly tension a bone or fracture void while maintaining reduction.

The features of a method of using the bone implant device may have similar advantages as the features of the tensioning bone implant device.

According to example embodiments of the present invention, a method of using a bone implant device may include inserting the device into a bone or fracture void such that the hinge, the first tensioning arm, and the second tensioning arm cooperate to tension the bone or fracture void. The bone implant device may include a resilient hinge, a first tensioning arm attached to one end of the hinge, and a second tensioning arm attached to an other end of the hinge, in which each of the hinge, the first tensioning arm, and the second tensioning arm includes at least one loop formed from wire.

The features of a method of manufacturing the bone implant device may have similar advantages as the features of the tensioning bone implant device.

According to example embodiments of the present invention, a method of manufacturing a bone implant device may include forming the resilient hinge by at least one loop, forming the first tensioning arm by at least one loop at the one end of the hinge, and forming the second tensioning arm by at least one loop at the other end of the hinge. The bone implant device may include a resilient hinge, a first tensioning arm attached to one end of the hinge, and a second tensioning arm attached to an other end of the hinge, in which each of the hinge, the first tensioning arm, and the second tensioning arm includes at least one loop formed from wire.

Example embodiments of the present invention are explained in greater detail in the following text with reference to the appended Figures.

### BRIEF DESCRIPTION OF THE DRAWINGS

FIG. 1 illustrates a schematic top view of a distal radial bone fracture.

FIG. 2 illustrates a schematic side view of a distal radial bone fracture.

FIG. 3 illustrates a schematic view along a longitudinal bone axis of an example embodiment of a tensioning bone implant device.

FIG. 4 illustrates a schematic perspective view of an example embodiment of a tensioning bone implant device.

FIG. 5 illustrates a view along a longitudinal bone axis of an example embodiment of a tensioning bone implant device.

FIG. 6 illustrates a partially formed example embodiment of a tensioning bone implant device.

### DETAILED DESCRIPTION

Exemplary embodiments of the tensioning bone implant device may restore metaphyseal and cortical collapse of bone or fracture voids, e.g., an extra-articular distal radial fracture, and thus may restore anatomic integrity. The device may offer a simple and minimally invasive alternative for unstable bone or fracture voids. For example, for unstable extra-articular distal radial fractures, the device may restore radial length and height and correct the radial shift, i.e., restore the radial column of a wrist.

Although the discussion herein may refer to unstable distal radial fractures, it is understood that the exemplary embodiments may apply equally to other types of bone or fracture voids.

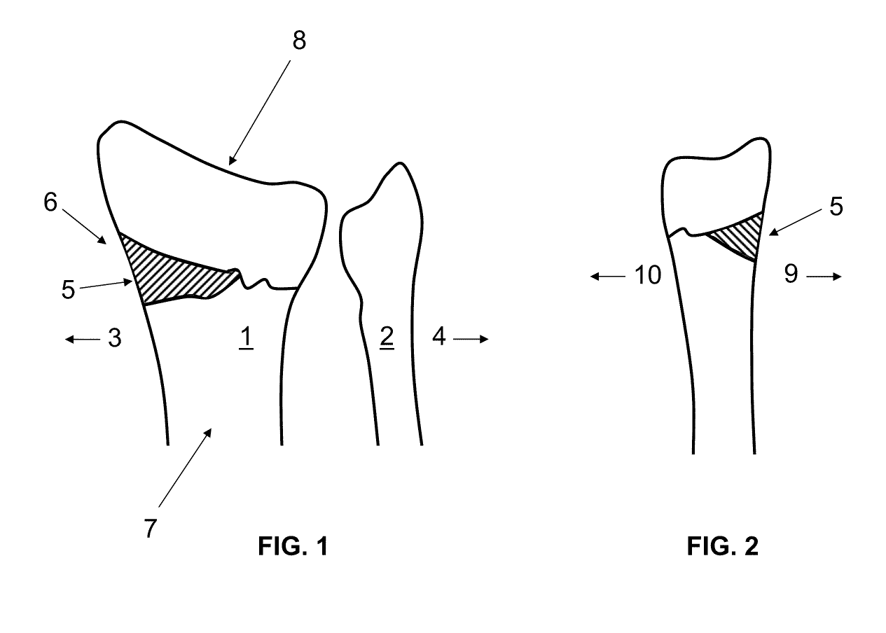

FIG. 1 illustrates a schematic top view of a distal radial bone fracture 5, e.g., viewed from a top of a right hand wrist. FIG. 1 illustrates a radius 1 and an ulna 2 of the right hand. The distal radial bone fracture 5 is situated on the radial side 3 of the radius 1 opposite the ulnar side 4. In addition, the distal radial bone fracture 5 is situated in the metaphysis 6 of the radius 1, between the diaphysis 7 and the epiphysis 8 of the radius 1. The bone fracture 5 as shown has created a metaphyseal void (shaded) on the radial side 3 of the radius 1.

FIG. 2 illustrates a schematic side view of a distal radial bone fracture 5, e.g., viewed from a thumb side of a right hand wrist. FIG. 2 illustrates only the radius 1, since the ulna 2 is substantially hidden behind the radius 1. The bone fracture 5 is situated on the dorsal side 9 of the radius 1 opposite the volar side 10. The bone fracture 5 as shown has created a metaphyseal void (shaded) on the dorsal side 9 of the radius 1.

The bone fracture 5 illustrated in FIGS. 1 and 2 is an example of an unstable, distal radius fracture that is extra-articular, i.e., the fracture is primarily outside of a joint. The fracture 5 as shown may result in dorsal comminution, i.e., pulverization of the bone on the dorsal side 9 of the radius 1. The fracture 5 as shown may also result in loss of radial height, i.e., loss of height of the bone on the radial side 3 of the radius 1. In addition, the fracture 5 as shown may result in loss of volar tilt, i.e., loss of tilt of the bone towards the volar side 10 of the radius 1. Further, the fracture 5 as shown may result in radial shift, i.e., shift of the bone towards the radial side 3 of the radius 1. Moreover, the fracture 5 as shown may result in shortening of the radial column.

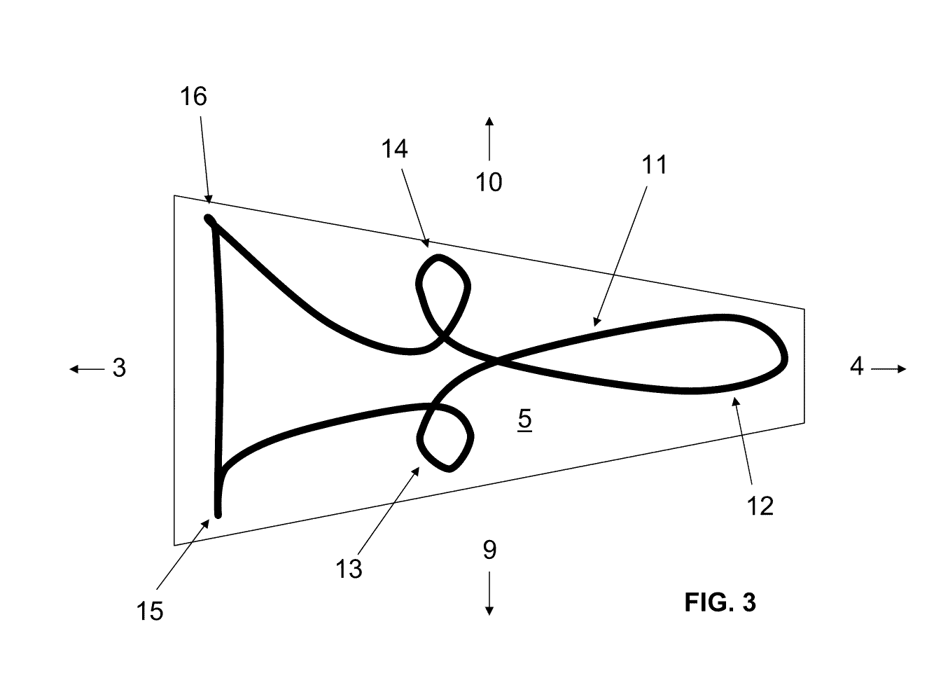

FIG. 3 illustrates a schematic view along a longitudinal bone axis of an example embodiment of a tensioning bone implant device 11. The bone implant device 11 is situated within the metaphyseal void created by bone fracture 5. The bone implant device 11 includes a resilient hinge 12 including at least one loop, a first tensioning arm 13 including at least one loop and attached to one end of the hinge 12, and a second tensioning arm 14 including at least one loop and attached to an other end of the hinge 12. The hinge 12 is situated on the ulnar side 4 of the fracture 5. The first tensioning arm 13 is situated on the dorsal side 9 of the fracture 5. The second tensioning arm 14 is situated on the volar side 10 of the fracture 5.

The exemplary bone implant device 11 as shown in FIG. 3 may also include a first seating element 15 at an end of the first tensioning arm 13 opposite the hinge 12. Further, the device 11 may also include a second seating element 16 at an end of the second tensioning arm 14 opposite the hinge 12. The seating elements 15, 16 may attach to bone on opposite sides of the fracture 5, e.g., the seating element 15 may attach on the dorsal side 9, and the seating element 16 may attach on the volar side 10. Further, seating element 15 may attach to a distal side of the fracture 5, and seating element 16 may attach to a proximal side of the fracture 5, or vice versa. In the example of the distal radial fracture of a wrist, distal side refers to a side nearer the hand, whereas proximal side refers to a side nearer the elbow.

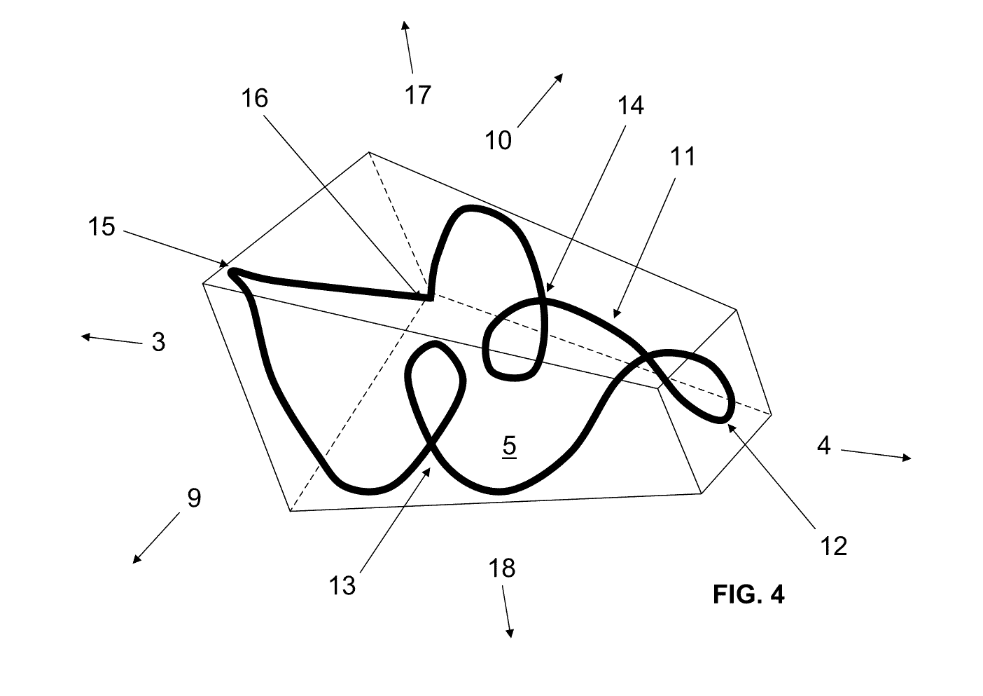

FIG. 4 illustrates a schematic perspective view of an example embodiment of a tensioning bone implant device 11. Similar to the example embodiment shown in FIG. 3, the bone implant device 11 is situated within the metaphyseal void created by bone fracture 5. The bone implant device 11 includes a resilient hinge 12 including at least one loop, a first tensioning arm 13 including at least one loop, and a second tensioning arm 14 including at least one loop. The hinge 12 is situated on the ulnar side 4 of the fracture 5. The first tensioning arm 13 is situated on the dorsal side 9 of the fracture 5. The second tensioning arm 14 is situated on the volar side 10 of the fracture 5.

The exemplary bone implant device 11 as shown in FIG. 4 may also include a first seating element 15 at an end of the first tensioning arm 13 opposite the hinge 12. Further, the device 11 may also include a second seating element 16 at an end of the second tensioning arm 14 opposite the hinge 12. The seating elements 15, 16 may attach to bone on opposite sides of the fracture 5, e.g., the seating element 15 may attach on the dorsal side 9, and the seating element 16 may attach on the volar side 10. Further, seating element 15 may attach to a distal side 17 of the fracture 5, and seating element 16 may attach to a proximal side 18 of the fracture 5, as shown, or vice versa.

As illustrated in FIGS. 3 and 4, the exemplary bone implant device 11 may form a substantially trapezoidal shape, or a substantially pyramidal shape. In addition, the device 11 may form other shapes, e.g., triangular, conical, oblong, ovoid, or others. Further, although the exemplary embodiments of FIGS. 3 and 4 include only a single loop for each of the hinge 12, the first tensioning arm 13, and the second tensioning arm 14, it is possible to include more than one loop in one or multiple of these components. In particular, the hinge 12 may include a single loop, whereas the first tensioning arm 13 and the second tensioning arm 14 each includes a double loop.

The bone implant device 11 may be formed from a single wire. That is, the hinge 12, the first tensioning arm 13, the second tensioning arm 14, the first seating element 15, and the second seating element 16 may be formed from a single wire. Alternatively, the elements of device 11 may be formed from two or more separate wires that are then joined to form the device 11. The wire of the device 11 may be formed from a biocompatible material. The biocompatible material may be stainless steel, nitinol, chrome-moly, other biocompatible metals, PEEK (polyaryletheretherketone), or other biocompatible polymers and plastics.

The device 11 may be pre-loaded with tension that distracts the radial column preferentially as a result of the looped wire, or coiled wire, configuration. Alternatively, the device 11 may be pre-loaded with tension via different configurations, e.g., a leaf spring configuration, or a ratcheted configuration. After implantation, the tensioning bone implant device 11 may provide sufficient resistance to compressive and torsional forces of hand usage during the fracture healing phase, which may typically last from 4 to 6 weeks.

The seating elements 15, 16 may include particular features, e.g., one or more loops, bumps, barbs, spurs, footholds, knuckles, or coils, which purchase the bone to which they are attached. In addition, the seating elements 15, 16 may be formed integrally with the wire forming each of the first tensioning arm 13 and the second tensioning arm 14, or the seating elements 15, 16 may be separate pieces joined to the first and second tensioning arms 13, 14 by any suitable joining method. For example, seating element 15, as shown in FIG. 4, may include a feature to attach to a distal side 17 of the fracture 5, and seating element 16, as shown in FIG. 4, may include a feature to attach to a proximal side 18 of the fracture 5. These seating elements 15, 16 may thus minimize shear or implant extrusion that would create fracture collapse, i.e., loss of reduction.

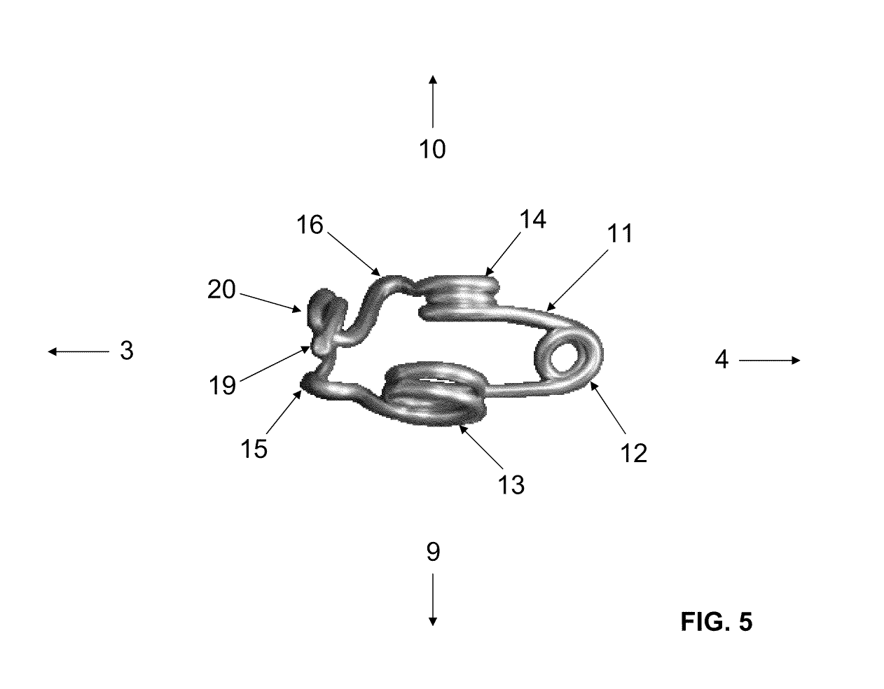

FIG. 5 illustrates a view along a longitudinal bone axis of an example embodiment of a tensioning bone implant device 11. Similar to the example embodiment shown in FIGS. 3 and 4, the example embodiment of FIG. 5 includes a hinge 12, a first tensioning arm 13, a second tensioning arm 14, a first seating element 15, and a second seating element 16. The hinge 12 is situated on the ulnar side 4 of the fracture 5, the first tensioning arm 13 is situated on the dorsal side 9 of the fracture 5, and the second tensioning arm is situated on the volar side 10 of the fracture 5.

In the example embodiment of FIG. 5, the hinge 12 includes a single small loop, the first tensioning arm 13 includes a double large loop, and the second tensioning arm 14 includes a double medium loop. The single small loop of the hinge 12 may be sized to fit within the ulnar side 4 of the fracture 5. Based on the exemplary distal radial fracture 5 illustrated in FIGS. 1 and 2, the fracture 5 on the dorsal side 9 is larger relative to the fracture 5 on the volar side 10. Thus, the double medium loop of the second tensioning arm 14 may be sized to fill the volar side 10 of the fracture 5, and the double large loop of the first tensioning arm 13 may be sized to fill the relatively larger dorsal side 9 of the fracture 5.

Although the exemplary embodiment of FIG. 5 includes either a single loop or double loops, it is understood that a variable number of loops may be utilized. The number of loops may be chosen to provide the desired spring effect and/or potentially better interference fit within the fracture 5. For example, an increase in the number of loops may increase the spring effect and/or improve the interference fit of the device 11. Moreover, the material of the device 11 and the thickness or gauge of the wire may be varied to achieve the desired spring effect and/or interference fit as well.

The hinge 12 may be configured such that the first tensioning arm 13 and the second tensioning arm 14 are biased away from each other. By this mechanism, the device 11 may provide additional tension to the fracture 5 and sufficiently fill the void, thereby acting as a three-dimensional reduction device and providing load-sharing. Because the tensioning bone implant device 11 itself acts as the reduction device, additional bone grafts or filler substitutes to fill the metaphyseal void may be unnecessary.

Further, the example embodiment of the device 11 shown in FIG. 5 includes a first locking element 19 and a second locking element 20. The first locking element 19 is situated on the first tensioning arm 13 at an end opposite the hinge 12, and the second locking element 20 is situated on the second tensioning arm 14 at an end opposite the hinge 12. The two locking elements 19, 20 may be configured to lock together to constrain a maximum distance between the first tensioning arm 13 and the second tensioning arm 14. For example, the locking elements 19, 20 may function similarly to a safety pin. In addition, the locking elements 19, 20 may be formed as hooks or any other shapes that may interlock or constrain relative movement.

When locked together, the two locking elements 19, 20 form an oblique radial strut between the seating elements 15, 16. That is, the seating elements 15, 16 and the locking elements 19, 20 may form a strut that traverses the radial side 3 of the fracture 5, e.g., from a dorsal, distal side 9, 17 of the fracture 5 to a volar, proximal side 10, 18 of the fracture 5. Alternatively, the strut may traverse the radial side 3 of the fracture 5, e.g., from a dorsal, proximal side 9, 18 of the fracture 5 to a volar, distal side 10, 17 of the fracture 5.

Moreover, as shown in the exemplary embodiment of the tensioning bone implant device 11 of FIG. 5, a plane formed by the at least one loop of the first tensioning arm 13 may be substantially parallel to a plane formed by the at least one loop of the second tensioning arm 14. In addition, a plane defined by the at least one loop of the hinge 12 may be substantially perpendicular to each of the plane defined by the at least one loop of the first tensioning arm 13 and the plane defined by the at least one loop of the second tensioning arm 14. This configuration may provide sufficient tension to, and filling of, the fracture 5.

Accordingly, the example embodiments of the tensioning bone implant device 11 may provide a one-piece device with universal sizing, depending on the ease of tailoring and locking in reduction. The device 11 may restore metaphyseal and cortical collapse of a bone or fracture void 5, e.g., an extra-articular distal radial fracture, by acting as a cortical strut and a three-dimensional reduction device filling the metaphyseal void. Thus, the device 11 may directly tension a bone or fracture void while maintaining reduction, thereby providing load-sharing and eliminating the need for additional bone grafts or other substitute fillers. Further, the device 11 may include slight over-distraction of the tensioning arms 13, 14 such that a bite of slight compression results when the device 11 is implanted into a bone or fracture void. Moreover, the device 11 may be implanted with minimal soft tissue invasion and bony disruption.

A method of using a bone implant device 11 may include inserting the device 11 into a bone or fracture void, such that the hinge 12, the first tensioning arm 13, and the second tensioning arm 14 cooperate to tension the bone or fracture void. As described herein, the bone implant device 11 may include a resilient hinge 12, a first tensioning arm 13 attached to one end of the hinge 12, and a second tensioning arm 14 attached to an other end of the hinge 12, in which each of the hinge 12, the first tensioning arm 13, and the second tensioning arm 14 includes at least one loop formed from wire.

The device 11 may be implanted via an incision providing access to the bone or fracture void 5. For example, for a distal radial fracture, the incision may be volar-radial, i.e., the Henry approach, or dorsal-radial between the first and second dorsal compartments. The fracture may be approached around the first dorsal compartment, releasing the distalmost fibers of the brachioradialis if necessary, and accommodating the instrumentation for distraction and reduction. This procedure may be performed with manual reduction. Although the exemplary embodiments disclosed herein refer to implanting the device 11 within a distal radial fracture, it is understood that the device 11 may be implanted in other bone fractures, voids, or defects of other bones.

For implantation of the device 11 within a distal radial bone or fracture void 5, the hinge 12 may be inserted first so that it becomes situated on an ulnar side 4 of the fracture 5, or the device 11 may be inserted in any other orientation. Because the hinge 12 biases the first and second tensioning arms 13, 14 away from each other, the arms 13, 14 may be compressed together during implantation in order to decrease the insertion profile of the device 11. During implantation, the first tensioning arm 13 may be oriented so that it is near the dorsal (larger) side 9 of the fracture, and the second tensioning arm 14 may be oriented so that it is near the volar (smaller) side 10 of the fracture. After the device 11 has been inserted into and properly located within the bone or fracture void, the compression on the arms 13, 14 may be released. Further, the device 11 may include slight over-distraction of the tensioning arms 13, 14 such that a bite of slight compression results when the device 11 is implanted into a bone or fracture void.

The device 11 may also include seating elements 15, 16, and locking elements 19, 20. After implantation, the seating elements 15, 16 may attach to bone adjacent the bone or fracture void, thereby securing the device 11 within the bone or fracture void. For example, the seating element 15 may attach to bone on a distal side 17 of the bone or fracture void, and the seating element 16 may attach to bone on a proximal side 18 of the bone or fracture void, or vice versa. In addition, locking elements 19, 20 may engage with each other in order to constrain a maximum distance between the first tensioning arm 13 and the second tensioning arm 14 within the bone or fracture void.

In addition, any suitable tools may be used to assist in the use of the device 11. Such tools may include, e.g., forceps, tweezers, clamps, applicators, and any specially-designed tools.

Accordingly, the device 11 may be implanted with minimal soft tissue invasion and bony disruption. Also, the device 11 may restore metaphyseal and cortical collapse of a bone or fracture void. Further, the device 11 may directly tension a bone or fracture void while maintaining reduction.

A method of manufacturing a bone implant device 11 may include forming the resilient hinge 12 by at least one loop, forming the first tensioning arm 13 by at least one loop at the one end of the hinge 12, and forming the second tensioning arm 14 by at least one loop at the other end of the hinge 12. As described herein, the bone implant device 11 may include a resilient hinge 12, a first tensioning arm 13 attached to one end of the hinge 12, and a second tensioning arm 14 attached to an other end of the hinge 12, in which each of the hinge 12, the first tensioning arm 13, and the second tensioning arm 14 includes at least one loop formed from wire.

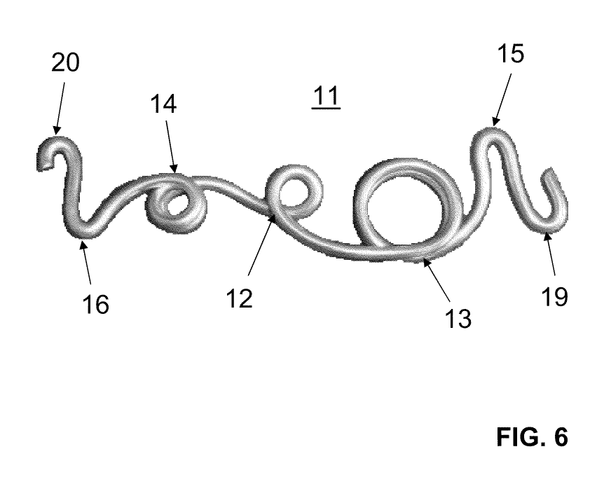

FIG. 6 illustrates a partially formed example embodiment of a tensioning bone implant device 11. As shown, the device 11 may be formed from a single wire that is bent and formed into each of the elements of the device 11. The wire may be composed of a biocompatible material. The biocompatible material may be stainless steel, nitinol, chrome-moly, other biocompatible metals, PEEK (polyaryletheretherketone), other biocompatible polymers and plastics, or other memory materials. Alternatively, the device 11 may be formed from multiple separate wires that are joined together.

As illustrated in FIG. 6, the hinge 12 is formed near the middle of the wire by at least one loop. The at least one loop of the hinge 12 may be a small single loop. The first tensioning arm 13 is formed on one end of the hinge 12, and the second tensioning arm 14 is formed on an other end of the hinge 12. The first tensioning arm 13 may include at least one loop, e.g., a large double loop. On an end of the first tensioning arm 13 opposite the hinge 12 there may be formed a first seating element 15 and a first locking element 19. The second tensioning arm 14 may include at least one loop, e.g., a medium double loop. On an end of the second tensioning arm 14 opposite the hinge 12 there may be formed a second seating element 16 and a second locking element 20.

The seating elements 15, 16 may be formed as one or more loops, bumps, barbs, spurs, footholds, knuckles, coils, or other features to secure the device 11 within a bone or fracture void. In addition, the locking elements 19, 20 may be formed as hooks or other features to interlock or constrain the free ends of the first and second tensioning arms 13, 14.

From the partially formed configuration shown in FIG. 6, the first and second tensioning arms 13, 14 may be further formed to result substantially in the shape of the device 11 shown in FIG. 5. That is, the first and second tensioning arms 13, 14 may be bent such that a plane formed by the at least one loop of the first tensioning arm 13 may be substantially parallel to a plane formed by the at least one loop of the second tensioning arm 14. In addition, the first and second tensioning arms 13, 14 may be bent such that a plane defined by the at least one loop of the hinge 12 may be substantially perpendicular to each of the plane defined by the at least one loop of the first tensioning arm 13 and the plane defined by the at least one loop of the second tensioning arm 14. As a result, the device 11 may substantially form a trapezoidal, pyramidal, triangular, conical, oblong, or ovoid shape. Moreover, the device 11 so formed may not require right- and left-handed configurations since the structure of the device 11 may readily lend itself to any configuration and/or orientation.

In addition to bending or mechanical manipulation of wire, the device 11 may be formed by other techniques or methods. For example, the device 11 may be formed by molding, extrusion, or any other techniques suitable for the particular material used.

Accordingly, the device 11 may be formed so as to be implanted with minimal soft tissue invasion and bony disruption. Also, the device 11 may be formed to restore metaphyseal and cortical collapse of a bone or fracture void. Further, the device 11 may be formed to directly tension a bone or fracture void while maintaining reduction.

What is claimed is:

1. A bone implant device, comprising:
a resilient hinge;
a first tensioning arm attached to one end of the hinge; and
a second tensioning arm attached to an other end of the hinge;
wherein each of the hinge, the first tensioning arm, and the second tensioning arm are formed of wire, and each of the hinge, the first tensioning arm, and the second tensioning arm includes at least one complete loop formed from the wire.

2. The bone implant device according to claim 1, wherein the hinge is configured to bias the first tensioning arm and the second tensioning arm away from each other.

3. The bone implant device according to claim 1, wherein the hinge includes at least a single loop.

4. The bone implant device according to claim 1, wherein the first tensioning arm includes at least a single loop.

5. The bone implant device according to claim 1, wherein the second tensioning arm includes at least a single loop.

6. The bone implant device according to claim 1, wherein a plane defined by the at least one loop of the first tensioning arm is substantially parallel to a plane defined by the at least one loop of the second tensioning arm.

7. The bone implant device according to claim 1, wherein a plane defined by the at least one loop of the hinge is substantially perpendicular to each of a plane defined by the at least one loop of the first tensioning arm and a plane defined by the at least one loop of the second tensioning arm.

8. The bone implant device according to claim 1, wherein the hinge, the first tensioning arm, and the second tensioning arm together substantially form one of a trapezoidal, pyramidal, triangular, conical, oblong, and ovoid shape.

9. The bone implant device according to claim 1, wherein the wire is composed of a biocompatible material.

10. The bone implant device according to claim 9, wherein the biocompatible materials includes at least one of stainless steel, nitinol, chrome-moly, other biocompatible metals, PEEK (polyaryletheretherketone), and other biocompatible polymers and plastics.

11. The bone implant device according to claim 1, further comprising a first locking element on the first tensioning arm at an end opposite the hinge.

12. The bone implant device according to claim 11, further comprising a second locking element on the second tensioning arm at an end opposite the hinge.

13. The bone implant device according to claim 12, wherein the first locking element and the second locking element are configured to lock together to constrain a maximum distance between the first tensioning arm and the second tensioning arm.

14. The bone implant device according to claim 1, further comprising a first seating element on the first tensioning arm at an end opposite the hinge, the first seating element configured to seat the first tensioning arm in bone on one side of a bone or fracture void.

15. The bone implant device according to claim 1, further comprising a second seating element on the second tensioning arm at an end opposite the hinge, the second seating element configured to seat the second tensioning arm in bone on an other side of a bone or fracture void.

16. The bone implant device according to claim 1, wherein the device is configured to be implanted with minimal soft tissue invasion and bony disruption.

17. The bone implant device according to claim 1, wherein the device is configured to restore metaphyseal and cortical collapse of a bone or fracture void.

18. The bone implant device according to claim 1, wherein the device is configured to directly tension a bone or fracture void while maintaining reduction.

19. A method of using a bone implant device, the bone implant device including a resilient hinge, a first tensioning arm attached to one end of the hinge, and a second tensioning arm attached to an other end of the hinge, the hinge, the first tensioning arm, and the second tensioning arm formed of wire, each of the hinge, the first tensioning arm, and the second tensioning arm including at least one complete loop formed from the wire, the method comprising:
inserting the device into a bone or fracture void such that the hinge, the first tensioning arm, and the second tensioning arm cooperate to tension the bone or fracture void.

20. A method of manufacturing a bone implant device, the bone implant device including a resilient hinge, a first tensioning arm attached to one end of the hinge, and a second tensioning arm attached to an other end of the hinge, the hinge, the first tensioning arm, and the second tensioning arm formed of wire, each of the hinge, the first tensioning arm, and the second tensioning arm including at least one complete loop formed from wire, the method comprising:
forming the resilient hinge by at least one loop;
forming the first tensioning arm by at least one loop at the one end of the hinge; and
forming the second tensioning arm by at least one loop at the other end of the hinge.
